(19) United States
(12) Patent Application Publication
(10) Pub. No.: US 2025/0218570 A1
(43) Pub. Date: Jul. 3, 2025

(54) INFORMATION PROCESSING SYSTEM, INFORMATION PROCESSING METHOD, AND PROGRAM

(71) Applicant: SONY GROUP CORPORATION, TOKYO (JP)

(72) Inventor: KEISUKE UYAMA, TOKYO (JP)

(21) Appl. No.: 18/849,525

(22) PCT Filed: Mar. 14, 2023
(86) PCT No.: PCT/JP2023/009780

§ 371 Date: Sep. 22, 2024

(30) Foreign Application Priority Data
Mar. 30, 2022 (JP) 2022-055041

(51) Int. Cl. G16H 30/20; G06V 20/40

(52) U.S. Cl. CPC G16H 30/20; G06V 20/47

(57) ABSTRACT

The present technology relates to an information processing system, an information processing method, and a program that enable only a necessary scene of a medical video to be easily uploaded to a server device.

A medical video captured by a medical image capturing device is acquired, a highlight scene that is a candidate to be preferentially uploaded to a storage is set on the basis of the medical video, a video of a first screen representing a range of images included in the highlight scene is generated on an arrangement screen in which images of frames at regular intervals in the medical video or images representative for each scene are arranged in chronological order, and the highlight scene is changed on the basis of a user operation.

### TECHNICAL FIELD

The present technology relates to an information processing system, an information processing method, and a program, and more particularly, to an information processing system, an information processing method, and a program capable of easily and quickly uploading only a necessary scene of a medical video to a server device.

### BACKGROUND ART

Patent Document 1 discloses a technique for reducing a burden of editing work of a user for shortening a long-time medical video (moving image file).

### CITATION LIST

### Patent Document

Patent Document 1: Japanese Patent Application Laid-Open No. 2019-185835

### SUMMARY OF THE INVENTION

### Problems to be Solved by the Invention

A long-time medical video is often captured for the purpose of recording, a surgical video or the like becomes a very long video, and it takes a lot of time to upload a high-quality video to a server or the like such as a cloud, and the memory capacity of the storage is consumed.

The present technology has been made in view of such a situation, and enables only a necessary scene of a medical video to be easily uploaded to a server device.

### Solutions to Problems

An information processing system or a program of the present technology is an information processing system including: an acquisition unit that acquires a medical video captured by a medical image capturing device; a setting unit that sets a highlight scene that is a candidate to be preferentially uploaded to a storage on the basis of the medical video; a display control unit that generates a video of a first screen representing a range of an image included in the highlight scene on an arrangement screen in which images of frames at regular intervals in the medical video or images representative for each scene are arranged in chronological order; and a change unit that changes the highlight scene on the basis of a user operation, or a program for causing a computer to function as such an information processing system.

An information processing method of the present technology is an information processing method including an acquisition unit, a setting unit, a display control unit, and a change unit, in which, in the information processing system, the acquisition unit acquires a medical video captured by a medical image capturing device, the setting unit sets a highlight scene that is a candidate to be preferentially uploaded to a storage on the basis of the medical video, the display control unit generates a video of a first screen representing a range of an image included in the highlight scene on an arrangement screen in which images of frames at regular intervals in the medical video or images representative for each scene are arranged in chronological order, and the change unit changes the highlight scene on the basis of a user operation.

In the information processing system, the information processing method, and the program of the present technology, a medical video captured by a medical image capturing device is acquired, a highlight scene that is a candidate to be preferentially uploaded to a storage is set on the basis of the medical video, a video of a first screen representing a range of images included in the highlight scene is generated on an arrangement screen in which images of frames at regular intervals in the medical video or images representative for each scene are arranged in chronological order, and the highlight scene is changed on the basis of a user operation.

### BRIEF DESCRIPTION OF DRAWINGS

FIG. 1 is a diagram schematically illustrating an overall configuration of an operating room system to which the technology according to the present disclosure can be applied.

FIG. 2 is a block diagram illustrating a configuration example of an information processing system according to an embodiment to which the present technology is applied.

FIG. 3 is a block diagram mainly illustrating a configuration example of an image processing device that uploads a video of a highlight scene to a cloud in the information processing system in FIG. 2.

FIG. 4 is a flowchart illustrating a procedure example of processing of the image processing device of FIG. 3.

FIG. 5 is a diagram illustrating a first form of a video observation screen.

FIG. 6 is a diagram illustrating an application example of a first form of a video observation screen.

FIG. 7 is a diagram illustrating a second form of a video observation screen.

FIG. 8 is a diagram illustrating a third form of a video observation screen.

FIG. 9 is a diagram illustrating a fourth form of a video observation screen.

FIG. 10 is a block diagram illustrating a configuration example of an embodiment of a computer to which the present technology is applied.

### MODE FOR CARRYING OUT THE INVENTION

Hereinafter, embodiments of the present technology will be described with reference to the drawings.

### Operating Room System to Which Present Technology is Applied

The technology according to the present disclosure can be applied to various products. For example, the technology according to the present disclosure may be applied to an operating room system.

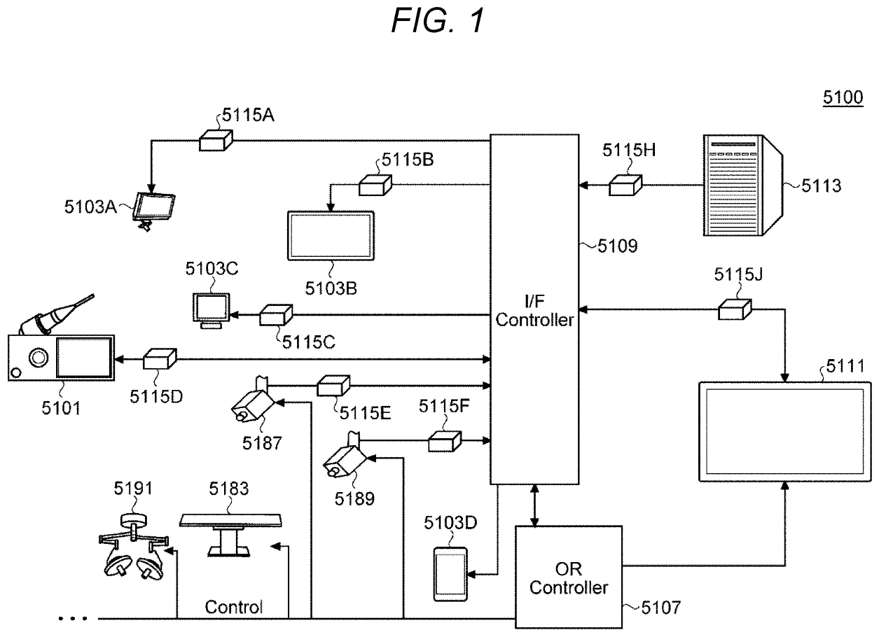

FIG. 1 is a diagram schematically illustrating an overall configuration of an operating room system 5100 to which the technology according to the present disclosure can be applied. Referring to FIG. 1, the operating room system 5100 is configured by connecting a group of devices installed in an operating room so as to be capable of cooperating with one another via an operating room (OR) controller 5107 and an interface controller (IF Controller) 5109. The operating room system 5100 is configured using an Internet Protocol (IP) network capable of transmitting and receiving 4K/8K images, and transmits and receives input and output images and control information for the devices via the IP network.

Various devices can be installed in the operating room. FIG. 1 illustrates, as examples, a group of various devices 5101 for endoscopic surgery, a ceiling camera 5187 that is provided on the ceiling of the operating room and captures an area near the hands of an operator, an operating field camera 5189 that is provided on the ceiling of the operating room and captures an overall situation in the operating room, a plurality of display devices 5103A to 5103D, a patient bed 5183, and a light 5191. In addition to an endoscope illustrated in Fig. B1, various medical devices for acquiring images and videos, such as a master-slave endoscopic surgery robot and an X-ray imaging device, may be applied to the group of devices 5101.

The group of devices 5101, the ceiling camera 5187, the operating field camera 5189, and the display devices 5103A to 5103C are connected to the IF controller 5109 via IP converters 5115A to 5115F (hereinafter, denoted by reference numeral 5115 when not individually distinguished). The IP converters 5115D, 5115E, and 5115F on video source sides (camera sides) perform IP conversion on videos from individual medical image capturing devices (such as an endoscope, an operation microscope, an X-ray imaging device, an operating field camera, and a pathological image capturing device), and transmit the results on the network. The IP converters 5115A to 5115D on video output sides (monitor sides) convert the videos transmitted through the network into monitor-unique formats, and output the results. The IP converters on the video source sides function as encoders, and the IP converters on the video output sides function as decoders. The IP converters 5115 may have various image processing functions, and may have functions of, for example, resolution conversion processing corresponding to output destinations, rotation correction and image stabilization of an endoscopic video, and object recognition processing. The image processing functions may also include partial processing such as feature information extraction for analysis on a server described later. These image processing functions may be specific to the connected medical image devices, or may be upgradable from outside. The IP converters on the display sides can perform processing such as synthesis of a plurality of videos (for example, picture-in-picture (PinP) processing) and superimposition of annotation information. The protocol conversion function of each of the IP converters is a function to convert a received signal into a converted signal conforming to a communication protocol allowing the signal to be transmitted on the network (such as the Internet). Any communication protocol may be set as the communication protocol. The signal received by the IP converter and convertible in terms of protocol is a digital signal, and is, for example, a video signal or a pixel signal. The IP converter may be incorporated in a video source side device or in a video output side device.

The group of devices 5101 belong to, for example, an endoscopic surgery system, and include, for example, the endoscope and a display device for displaying an image captured by the endoscope. The display devices 5103A to 5103D, the patient bed 5183, and the light 5191 are, for example, devices equipped in the operating room separately from the endoscopic surgery system. Each of these devices for surgical or diagnostic is also called a medical device. The OR controller 5107 and/or the IF controller 5109 controls operations of the medical devices in cooperation. When the endoscopic surgery robot (surgery master-slave) system and the medical image acquisition devices such as an X-ray imaging device are included in the operating room, those devices can also be connected as the group of devices 5101 in the same manner.

The OR controller 5107 controls processing related to image display in the medical devices in an integrated manner. Specifically, the group of devices 5101, the ceiling camera 5187, and the operating field camera 5189 among the devices included in the operating room system 5100 can each be a device having a function to transmit (hereinafter, also called a transmission source device) information to be displayed (hereinafter, also called display information) during the operation. The display devices 5103A to 5103D can each be a device to output the display information (hereinafter, also called an output destination device). The OR controller 5107 has a function to control operations of the transmission source devices and the output destination devices so as to acquire the display information from the transmission source devices and transmit the display information to the output destination devices to cause the output destination devices to display or record the display information. The display information refers to, for example, various images captured during the operation and various types of information on the operation (for example, body information and past examination results of a patient and information about a surgical procedure).

Specifically, information about an image of a surgical site in a body cavity of the patient captured by the endoscope can be transmitted as the display information from the group of devices 5101 to the OR controller 5107. Information about an image of the area near the hands of the operator captured by the ceiling camera 5187 can be transmitted as the display information from the ceiling camera 5187. Information about an image representing the overall situation in the operating room captured by the operating field camera 5189 can be transmitted as the display information from the operating field camera 5189. When another device having an imaging function is present in the operating room system 5100, the OR controller 5107 may also acquire information about an image captured by the other device as the display information from the other device.

The OR controller 5107 displays the acquired display information (that is, the images captured during the operation and the various types of information on the operation) on at least one of the display devices 5103A to 5103D serving as the output destination devices. In the illustrated example, the display device 5103A is a display device installed on the ceiling of the operating room, being hung therefrom; the display device 5103B is a display device installed on a wall surface of the operating room; the display device 5103C is a display device installed on a desk in the operating room; and the display device 5103D is a mobile device (such as a tablet personal computer (PC)) having a display function.

The IF controller 5109 controls input and output of the video signal from and to connected devices. For example, the IF controller 5109 controls input and output of the video signal based on controlling of the OR controller 5107. The IF controller 5109 includes, for example, an IP switcher, and controls high-speed transfer of the image (video) signal between devices disposed on the IP network.

The operating room system 5100 may include a device outside the operating room. The device outside the operating room can be a server connected to a network built in and outside a hospital, a PC used by a medical staff, or a projector installed in a meeting room of the hospital. When such an external device is present outside the hospital, the OR controller 5107 can also display the display information on a display device of another hospital via, for example, a teleconference system for telemedicine.

An external server 5113 is, for example, an in-hospital server or a cloud server outside the operating room, and may be used for, for example, image analysis and/or data analysis. In this case, the video information in the operating room may be transmitted to the external server 5113, and the server may generate additional information through big data analysis or recognition/analysis processing using artificial intelligence (AI) (machine learning), and feed the additional information back to the display devices in the operating room. At this time, an IP converter 5115H connected to the video devices in the operating room transmits data to the external server 5113, so that the video is analyzed. The transmitted data may be, for example, a video itself of the operation using the endoscope or other tools, metadata extracted from the video, and/or data indicating an operating status of the connected devices.

The operating room system 5100 is further provided with a central operation panel 5111. Through the central operation panel 5111, a user can give the OR controller 5107 an instruction about input/output control of the IF controller 5109 and an instruction about an operation of the connected devices. Furthermore, the user can switch image display via the central operation panel 5111. The central operation panel 5111 is configured by providing a touchscreen on a display surface of a display device. The central operation panel 5111 may be connected to the IF controller 5109 via an IP converter 5115J.

The IP network may be established using a wired network, or a part or the whole of the network may be established using a wireless network. For example, each of the IP converters on the video source sides may have a wireless communication function, and may transmit the received image to an output side IP converter via a wireless communication network, such as the fifth-generation mobile communication system (5G) or the sixth-generation mobile communication system (6G).

### Information Processing System According to Present Embodiment

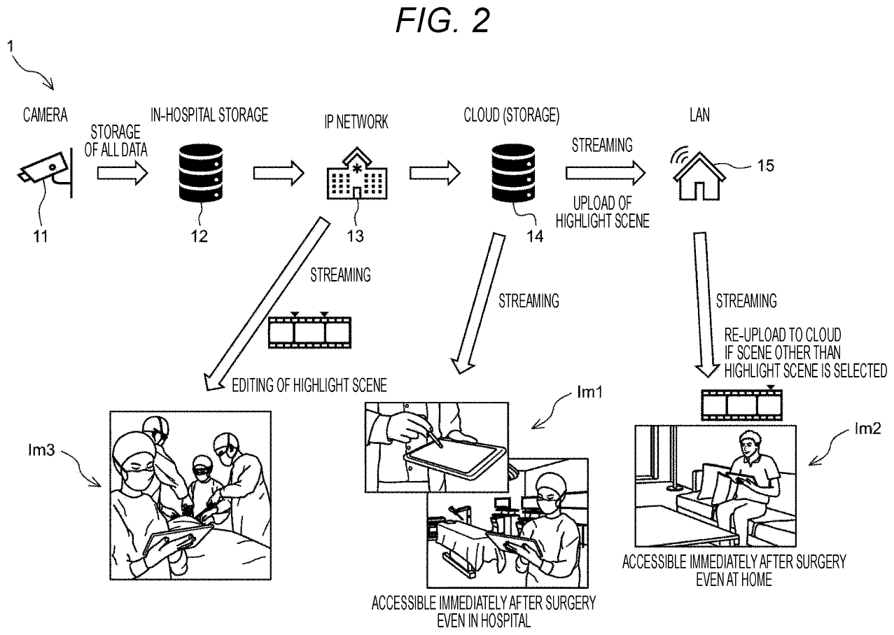

FIG. 2 is a block diagram illustrating a configuration example of an information processing system according to an embodiment to which the present technology is applied.

In FIG. 2, the information processing system 1 according to the present embodiment includes a camera 11, an in-hospital storage 12, an IP network 13, a cloud 14, and a local area network (LAN) 15.

The camera 11 corresponds to any one of the medical image capturing devices (endoscope, operation microscope, x-ray imaging device, operating field camera, pathological image capturing device, or the like) in FIG. 1. The vide (medical video) captured by the camera 11 is supplied to the in-hospital storage 12.

The in-hospital storage 12 is a storage connected to the IP network 13 of FIG. 2 corresponding to the IP network of FIG. 1, or a storage in which data is read and written via a device connected to the IP network. The in-hospital storage 12 temporarily stores the video captured by the camera 11. The video stored in the in-hospital storage 12 is supplied to the IP network 13.

The IP network 13 corresponds to the IP network of FIG. 1, and supplies (uploads) the video from the in-hospital storage 12 to the storage of the cloud 14 connected to the IP network.

The cloud 14 corresponds to the external server 5113 in FIG. 1. The cloud 14 is a form of technology in which one or a plurality of server devices is shared and used by a plurality of users, and is not limited thereto, and may be a server device including a storage that stores data such as videos or may be an in-hospital server (server device) outside the operating room. The cloud 14 permanently stores (for storage) the video from the in-hospital storage 12 uploaded via the IP network 13. Note that the video temporarily stored in the in-hospital storage 12 is deleted when an unused state elapses for a certain period of time or more. The video uploaded to the cloud 14 can be viewed by a terminal device connected to the cloud 14 via a communication network such as the Internet or a wide area network (WAN) as indicated by an image picture Im1 on the lower side of FIG. 2. Since a video captured by the camera 11 can be uploaded to the cloud 14 in real time, for example, in a case where a medical video (surgical video) is uploaded to the cloud 14, the medical video can be viewed on the terminal device immediately after surgery. Note that audio as well as video can be uploaded to the cloud 14, but only video is uploaded to the cloud 14. A case where only a video is viewed is also referred to as viewing.

The LAN 15 is a local communication network connected to the cloud 14 via a communication network such as the Internet or a wide area network (WAN), for example, and represents a communication network different from the IP network 13. The video uploaded to the cloud 14 as indicated by an image picture Im2 at the lower left of FIG. 2 can be viewed on a terminal device connected to the LAN 15 via a communication network such as the Internet. Therefore, it is possible to view the video uploaded to the cloud 14 even in a place outside the hospital such as a house.

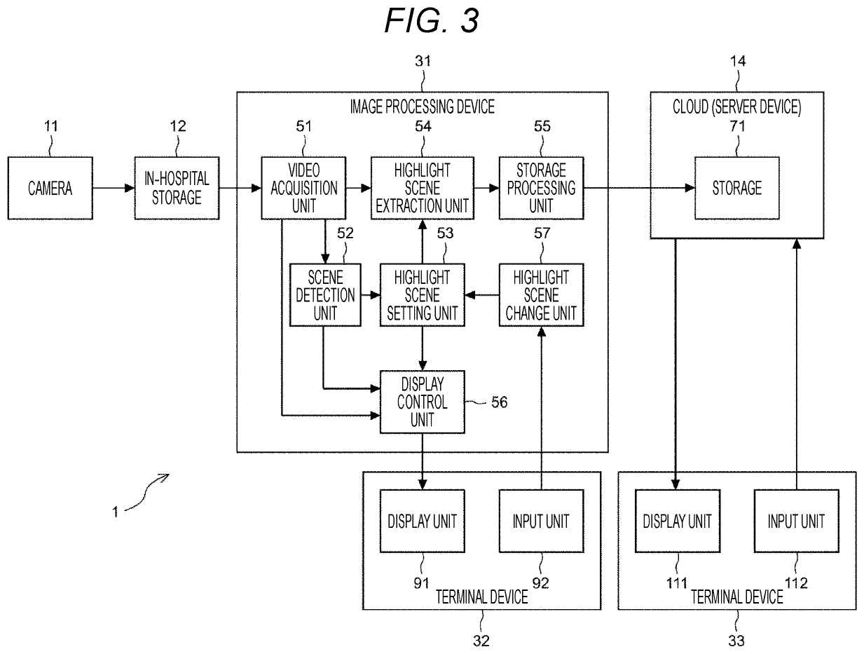

As described with reference to FIG. 3, the video temporarily stored in the in-hospital storage 12 is captured by the image processing device connected to the IP network of FIG. 1 via the IP network 13, and the video of the specific scene is automatically extracted as the video of the highlight scene. The video of the highlight scene extracted by the image processing device is uploaded to the cloud 14 via the IP network 13 as a high-resolution video. As a result, since only a part of the video captured by the camera 11 is uploaded to the cloud 14 with high resolution, the time required for uploading is shortened, the memory resources in the cloud 14 are saved, and the video of only the necessary portion can be viewed on the terminal device outside the operating room.

Furthermore, the image processing device can supply the video of the video observation screen to the terminal device connected to the IP network 13 by, for example, streaming and display the video. The video observation screen includes a main screen and a highlight scene editing screen. Note that the main screen and the highlight scene editing screen are assumed to be displayed together as one screen, but only one of the main screen and the highlight scene editing screen may be switchably displayed. The main screen is a screen that presents the real-time video captured by the camera 11 captured by the image processing device from the in-hospital storage 12 to the user. The highlight scene editing screen is a screen for the user to confirm the contents of the video of the highlight scene uploaded (or candidates to be uploaded) to the storage 71 of the cloud 14 and to change the temporal range (also referred to as a highlight scene range) of the video to be the highlight scene as necessary. The terminal device 32 connected to the IP network 13 corresponds to, for example, the display devices 5103A to 5103D or the central operation panel 5111 in FIG. 1. An image picture Im3 at the lower left of FIG. 2 illustrates a state in which an operator performing an operation or an assistant other than the operator (hereinafter, referred to as a user) is checking an upload range on a terminal device (tablet PC), and the user can edit (change) the highlight scene range as necessary by an operation on the terminal device.

### Block Diagram of Information Processing System 1

FIG. 3 is a block diagram mainly illustrating a configuration example of an image processing device that uploads a video of a highlight scene captured by the camera 11 to the cloud 14 in the information processing system 1 of FIG. 2. Note that, in the drawing, the same reference signs are given to portions common to those in FIG. 2, and the description thereof will be omitted as appropriate. Furthermore, in the drawing, a configuration regarding communication between the devices is omitted.

In FIG. 3, the information processing system 1 includes a camera 11, an in-hospital storage 12, a cloud 14, an image processing device 31, a terminal device 32, and a terminal device 33.

The camera 11 and the in-hospital storage 12 correspond to the camera 11 and the in-hospital storage 12 in FIG. 2. The cloud 14 corresponds to the cloud 14 in FIG. 2, and the cloud 14 has a storage 71 that stores data and can read the stored data.

In FIG. 2, the terminal device 32 corresponds to a terminal device (for example, a tablet PC) connected to the IP network 13. The terminal device 32 includes a display unit 91 that displays a video (image) and an input unit 92 to which a user's operation is input. The input unit 92 may be, for example, a touch panel or the like installed on a screen surface of the display unit 91. Furthermore, the input unit 92 may be a case where a voice of the user is input.

The terminal device 33 is a terminal device connected to the cloud 14 via a communication network in FIG. 2. The terminal device 33 represents an arbitrary terminal device connected to the cloud 14 without the IP network 13. For example, the terminal device 33 is a terminal device connected to the cloud via the LAN 15 or a terminal device connected to the cloud 14 without the LAN 15. The terminal device 33 includes a display unit 111 that displays a video (image) and an input unit 112 to which a user's operation is input. The input unit 112 may be, for example, a touch panel or the like installed on a screen surface of the display unit 91. The input unit 112 may be a case where a user's voice is input.

The image processing device 31 may be incorporated in an arbitrary IP converter 5115 in FIG. 1, may be incorporated in an arbitrary device connected to the IP network 13, or may be an independent device connected to the IP network 13. The image processing device 31 includes a video acquisition unit 51, a scene detection unit 52, a highlight scene setting unit 53, a highlight scene extraction unit 54, a storage processing unit 55, and a display control unit 56.

The video acquisition unit 51 acquires the video data temporarily stored in the in-hospital storage 12 in chronological order. The video data includes image data of a plurality of frames captured at times at regular intervals. In addition, a time code indicating the imaging time is added to the image data of each frame or the image data of frames at regular intervals. The video acquisition unit 51 acquires the video data (image data of frames) stored in the in-hospital storage 12 from the camera 11 in chronological order of imaging time, and acquires the video data imaged by the camera 11 substantially at the same time as imaging (in real time). Furthermore, in a case where the highlight scene range uploaded as the video data of the highlight scene is changed by the user's operation, the video acquisition unit 51 acquires the video data of the highlight scene range from the in-hospital storage 12. The video acquisition unit 51 supplies the real-time video data acquired from the in-hospital storage 12 to the scene detection unit 52.

The scene detection unit 52 detects scene switching from the real-time video data from the video acquisition unit 51, and acquires a time code (imaging time) of a frame when the switching is detected. As a result, the scene detection unit 52 detects, as one scene, a video from the imaging time (start time) of the frame when the scene switching is detected to the imaging time (end time) of the frame when the scene switching is detected next. Information of a time code indicating a time range (start time and end time) of each scene and video data are supplied to the highlight scene setting unit 53.

The highlight scene setting unit 53 sets a scene that satisfies a predetermined condition as a highlight scene on the basis of the video data of each scene from the scene detection unit 52. The highlight scene means, for example, a scene considered to be important during surgery among videos from before the start of the surgery to after the end of the surgery. The highlight scene may be a case where the highlight scene is specified on the basis of a brightness change of a scene or voice recognition (such as an explicit operation scene instruction of a staff member). In addition, the type of the highlight scene may be recognized using machine learning (inference model), or the type of the scene may be determined by structure recognition of a person or a hand to determine whether or not the scene is a highlight scene. The highlight scene setting unit 53 sets the automatically set highlight scene as the highlight scene of the standard setting. Furthermore, in a case where the highlight scene change unit 57 (described later) specifies a change of the highlight scene based on the user's operation, the highlight scene setting unit 53 changes the highlight scene to be set from the standard settings to the scene in the time range specified by the highlight scene change unit 57. The highlight scene setting unit 53 sets the highlight scene designated by the highlight scene change unit 57 as a highlight scene of the user setting. The highlight scene setting unit 53 supplies time code information indicating the time range of the highlight scene of the standard setting (referred to as highlight scene information of the standard setting) and time code information indicating the time range of the highlight scene of the user setting (referred to as highlight scene information of the user setting) to the highlight scene extraction unit 54. Note that, when setting the start time of the highlight scene for the real-time video data acquired by the video acquisition unit 51, the highlight scene setting unit 53 supplies the information to the highlight scene extraction unit 54 as highlight scene information. When setting the end time of the highlight scene for the real-time video data acquired by the video acquisition unit 51, the highlight scene setting unit 53 supplies the information as highlight scene information to the highlight scene extraction unit 54.

The highlight scene extraction unit 54 extracts video data (image data of a frame) of a time range of the highlight scene of the standard setting from among the real-time video data acquired by the video acquisition unit 51 on the basis of the highlight scene information of the standard setting. At this time, in a case where the highlight scene continues after the start time of the highlight scene of the standard setting at the latest imaging time of the real-time video data acquired by the video acquisition unit 51 (in a case where the end time is not given from the highlight scene setting unit 53), the highlight scene extraction unit 54 extracts the video data from the start time of the highlight scene of the standard setting to the latest imaging time. As time passes, the imaging time of the latest video data (image data of a frame) acquired by the video acquisition unit 51 also advances, and when information on the end time of the highlight scene of the standard setting is given from the highlight scene setting unit 53, the highlight scene extraction unit 54 extracts the video data up to the end time from the video acquisition unit 51, and ends the extraction of the video data of the highlight scene. The highlight scene extraction unit 54 supplies the video data of the extracted highlight scene of the standard setting to the storage processing unit 55.

Furthermore, in a case where the highlight scene information of the user setting is given, the highlight scene extraction unit 54 extracts the video data of the time range of the highlight scene of the user setting from the video acquisition unit 51 on the basis of the information, similarly to the extraction of the video data of the highlight scene of the standard setting. However, the highlight scene of the user setting is set such that after the highlight scene of the standard setting is set by the highlight scene setting unit 53, a change is made to the standard settings. Therefore, the past time may be the start time or end time of the highlight scene with respect to the latest imaging time of the real-time video data acquired by the video acquisition unit 51. In that case, the highlight scene extraction unit 54 instructs the video acquisition unit 51 to acquire the video data of the time range of the highlight scene of the user setting from the in-hospital storage 12 again, and supplies the video data of the time range to the storage processing unit 55.

The storage processing unit 55 encodes (compresses) the video data of the highlight scene from the highlight scene extraction unit 54 into video data of a predetermined format. At this time, the storage processing unit 55 generates high-resolution video data by encoding the video data of the highlight scene at a low compression rate. The storage processing unit 55 uploads the encoded video data of the highlight scene to the cloud 14 (storage 71). Further, when the video data of the highlight scene of the standard setting is being uploaded to the cloud 14, or in a case where the highlight scene of the standard setting is changed to the highlight scene of the user setting after being uploaded, the video data of the highlight scene of the standard setting is deleted from the storage 71 of the cloud 14, and the video data of the highlight scene of the user setting is uploaded to the cloud 14 (the storage 71). However, the video data of the highlight scene of the standard setting may be integrated with the video data of the highlight scene of the user setting without being deleted from the storage 71 of the cloud 14. For example, when uploading the video data of the highlight scene of the user setting, the storage processing unit 55 deletes the video data of the time range not included in the highlight scene of the user setting from the cloud 14 among the video data of the highlight scene of the standard setting already uploaded to the cloud 14, and leaves the video data of the time range included in the highlight scene of the user setting in the cloud 14. Then, the storage processing unit 55 uploads only the video data of the time range not uploaded to the cloud 14 among the video data of the highlight scene of the user setting to the cloud 14, and combines the video data with the video data uploaded to the cloud 14. In addition, the storage processing unit 55 may upload the video data of high resolution of all scenes to the cloud 14 without limiting to the video data of the highlight scene, and may delete the video data other than the highlight scene from the cloud 14 after a certain period of time has elapsed. Processing such as deletion of video data in the cloud 14 is not limited to being performed by an instruction from the image processing device 31 such as the storage processing unit 55 to the cloud 14, and may be performed by determination processing in the cloud 14 by the cloud 14 acquiring information such as a time range of a highlight scene. In addition, all the video data acquired by the camera 11 may be temporarily stored in an arbitrary storage in the hospital such as the in-hospital storage 12, and the image processing device 31 may upload the video data of the highlight scene of the standard setting or the user setting to the cloud 14 according to a request from the terminal device such as the terminal device 32 or the terminal device 33 not in real time with the imaging by the camera 11 but after the imaging is finished.

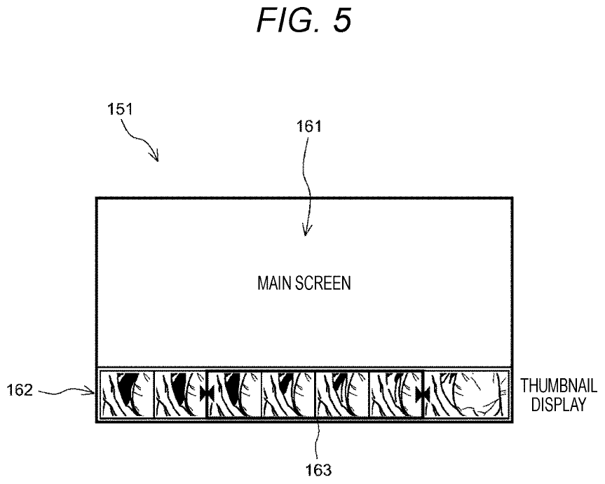

Furthermore, the storage processing unit 55 acquires video data of a scene (non-highlight scene) other than the highlight scene from the video acquisition unit 51, and encodes the video data of the non-highlight scene with a higher compression rate than the video data of the highlight scene, thereby generating video data (video data of proxy video) with low resolution (low data amount). The storage processing unit 55 uploads the encoded video data of the non-highlight scene to the storage 71 of the cloud 14. However, the generation of the low-resolution video data is not limited to a case where encoding is performed at a high compression rate, and may be a case where the video size (the number of vertical and horizontal pixels) is reduced. The encoding of the video data includes changing the video size of the video data, and the encoding of the high compression rate includes a case where the video size is reduced. Furthermore, encoding of the video data of the non-highlight scene and upload to the cloud 14 are performed when encoding of the video data of the highlight scene and upload to the cloud 14 are not performed, respectively, and encoding of the video data of the highlight scene and upload to the cloud 14 are preferentially performed. Furthermore, in a case where a limit is imposed on the amount of data that can be uploaded to the storage 71 of the cloud 14, the storage processing unit 55 prioritizes encoding the video data of the highlight scene at a certain compression rate or less, and performs encoding while adjusting the compression rate of the video data of the non-highlight scene, so that the data amount of the video data to be uploaded to the storage 71 of the cloud 14 falls within the limit range. That is, the storage processing unit 55 uploads the video data of the highlight scene to the cloud 14 in preference to the video data of the non-highlight scene in terms of time (order) and image quality. Note that the video data uploaded to the cloud 14 as the video data of the non-highlight scene may be a thumbnail (reduced image) displayed on the highlight scene editing screen in the video observation screen described in FIG. 5 and the like. The thumbnail of the image included in the highlight scene may also be uploaded to the cloud 14. In addition, the video data of the highlight scene may be uploaded to the cloud 14 as the high-resolution video data, and all the video data of the highlight scene and the non-highlight scene may be uploaded to the cloud 14 as the low-resolution video data, that is, as the video data of the proxy video. Furthermore, the video data of the non-highlight scene may not be uploaded to the cloud 14, and only the video data of the highlight scene may be uploaded to the cloud 14. In addition, together with the video data, information regarding a highlight scene range (information specifying a time range of a highlight scene (video data to be uploaded), or the like) and information regarding a scene (information detected by the scene detection unit 52, or the like) may be uploaded to the cloud 14 as metadata, and the metadata may be used when distributing the video from the cloud 14 to the terminal device 33.

As described above, the display control unit 56 generates the video of the video observation screen to be displayed on the terminal device 32 and presented to the user. The video observation screen includes a main screen and a highlight scene editing screen. Note that the main screen and the highlight scene editing screen are assumed to be displayed together as one screen, but only one of the main screen and the highlight scene editing screen may be switchably displayed. The main screen is a screen that presents the real-time video captured by the camera 11 captured by the video acquisition unit 51 from the in-hospital storage 12 to the user. The highlight scene editing screen is a screen for the user to confirm the contents of the video of the highlight scene uploaded to the storage 71 of the cloud 14 (or candidates to be uploaded) and to change the highlight scene range as necessary. A specific example of the video observation screen will be described later. The display control unit 56 supplies the generated video of the video observation screen to the terminal device 32 by, for example, streaming and causes the display unit 91 to display the video. In the terminal device 32, when the user inputs an operation of changing the highlight scene range from the input unit 92 to the video observation screen displayed on the display unit 91, the operation of the user is supplied to the highlight scene change unit 57. Note that the user's operation for changing the highlight scene range may be performed by voice.

The highlight scene change unit 57 sets the time range of the highlight scene of the user setting on the basis of the user's operation input from the input unit 92 on the video observation screen displayed on the display unit 91 of the terminal device 32. The highlight scene change unit 57 designates the time range of the set highlight scene of the user setting to the highlight scene setting unit 53. Note that the highlight scene change unit 57 may set the time range of the highlight scene of the user setting on the basis of the operation of the user not from the terminal device 32 but from the terminal device 33.

The video data of the highlight scene stored in the storage 71 of the cloud 14 is supplied (distributed) to the terminal device 33 connected to the cloud 14 by streaming or the like and displayed on the display unit 111. In addition, a video similar to the highlight scene editing screen displayed on the terminal device 33 is generated by the cloud 14 by using the video data (and metadata) of the highlight scene and the non-highlight scene stored in the storage 71, and is displayed on the display unit 111 of the terminal device 33. When the user inputs an operation of changing the highlight scene range from the input unit 112 on the editing screen, the operation is transmitted to the cloud 14. When the operation to change the highlight scene range is performed, the cloud 14 designates the time range after the change of the highlight scene designated by the user for the highlight scene setting unit 53 of the image processing device 31. As a result, the highlight scene setting unit 53 sets the time range of the highlight scene designated from the cloud 14 as the time range of the highlight scene of the user setting, so that the video data of the highlight scene of the user setting is uploaded to the cloud 14. The video date of the highlight scene of the user setting newly uploaded to the cloud 14 can be displayed on the display unit 111 of the terminal device 33.

### Processing Procedure Example of Image Processing Device 31

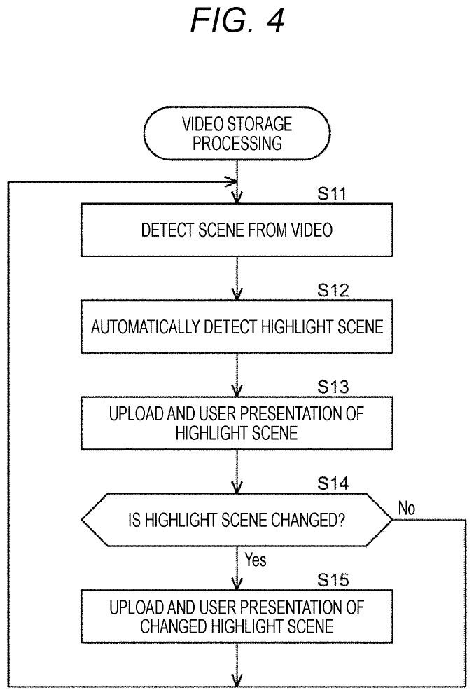

FIG. 4 is a flowchart illustrating a procedure example of processing of the image processing device 31 of FIG. 3. In step S11, real-time video data captured by the camera 11 is supplied from the video acquisition unit 51 to the scene detection unit 52 of the image processing device 31, and the scene detection unit 52 detects a scene (a start time and an end time of each scene) by detecting scene switching from the supplied real-time video data. The process proceeds from step S11 to step S12.

In step S12, the highlight scene setting unit 53 detects a scene satisfying a predetermined condition among the scenes detected by the scene detection unit 52 in step S11, and sets the scene as a highlight scene. The process proceeds from step S12 to step S13. In step S13, the highlight scene extraction unit 54 extracts the video data of the highlight scene of the standard setting set by the highlight scene setting unit 53 in step S12 from the real-time video data acquired by the video acquisition unit 51. The storage processing unit 55 encodes the video data of the highlight scene of the standard setting extracted by the highlight scene extraction unit 54 at a low compression rate, and uploads the high-resolution video data to the cloud 14. In addition, the display control unit 56 generates a main screen for presenting a real-time video and a video of a highlight scene editing screen for the user to confirm the video content of the highlight scene of the standard setting and to change the highlight scene range as necessary, and displays the generated video as a video observation screen on the display unit 91 of the terminal device 32 connected to the IP network 13. The process proceeds from step S13 to step S14.

In step S14, the highlight scene change unit 57 detects a user operation input from the input unit 92 of the terminal device 32, and determines whether or not an instruction to change the highlight scene range of the standard settings has been issued. In a case of negative in step S14, the process skips step S15 and returns to step S11. In the case of affirmative determination in step S14, the process proceeds to step S15.

In step S15, the highlight scene setting unit 53 sets the time range of the highlight scene changed by the user operation from the highlight scene change unit 57 as the time range of the highlight scene of the user setting (changed). Similarly to step S13, the video data of the highlight scene of the user setting set by the highlight scene setting unit 53 is uploaded to the cloud 14 as high-resolution video data, and the video of the highlight scene editing screen in which the video content of the highlight scene of the user setting is presented is displayed on the display unit 91 of the terminal device 32. After step S15, the process returns to step S11, and is repeated from step S11.

According to the procedure example of FIG. 4, since the video data of the highlight scene is uploaded to the cloud 14 in the background regardless of the presence or absence of the user's operation, the video data is efficiently uploaded. Note that, in the procedure example of FIG. 4, the video data of the highlight scene of the standard setting is automatically uploaded to the cloud 14 in step S13, but the video data of the highlight scene may be uploaded to the cloud 14 only in a case where the upload instruction operation is explicitly performed by the user after confirmation of the video data of the highlight scene of the standard setting, setting of the highlight scene of the user setting in step S14, or the like. Furthermore, although omitted in the procedure example of FIG. 4, the storage processing unit 55 may appropriately encode the video data of the non-highlight scene or the video data of all scenes of the highlight scene and the non-highlight scene at a high compression rate except when encoding is performed at a low compression rate on the video data of the highlight scene in step S13 or step S15. Furthermore, the storage processing unit 55 may appropriately upload the low-resolution video data of the non-highlight scene or the low-resolution video data of all the scenes of the highlight scene and the non-highlight scene to the cloud 14 except when the high-resolution video data of the highlight scene is uploaded to the cloud 14 in step S13 or step S15.

### First Form of Video Observation Screen

FIG. 5 is a diagram illustrating a first form of a video observation screen generated by the display control unit 56 of the image processing device 31 in FIG. 3 and displayed on the display unit 91 of the terminal device 32. In FIG. 5, a video observation screen 151 of the first form includes a main screen 161 and a highlight scene editing screen 162.

A real-time video captured by the camera 11 is displayed on the main screen 161. On the highlight scene editing screen 162, thumbnails of images of frames at regular time intervals in the video captured by the camera 11, or thumbnails representing each scene (such as the thumbnail at the head of the frame) are displayed in order from left to right in chronological order of the imaging time. In a case where a thumbnail representing each scene is displayed on the highlight scene editing screen 162, a video obtained by frame advance of the scene may be used as the thumbnail. In addition, these display forms of the highlight scene editing screen 162 may be switchable (hereinafter, it similarly applies to other forms). A highlight scene range frame 163 is displayed on the highlight scene editing screen 162. The highlight scene range frame 163 is a frame image surrounding a thumbnail of a video (a video that is a candidate to be uploaded) captured within the time range of the highlight scene. Note that a filter of a predetermined color may be superimposed and displayed on the thumbnail in the highlight scene range frame 163, or the thumbnail in the highlight scene range frame 163 may be a color image and the thumbnail outside the highlight scene range may be a black-and-white image (grayscale image). The range of the highlight scene range frame 163 is initially set on the basis of the time range of the highlight scene of the standard setting. On the other hand, the range of the highlight scene range frame 163 can be changed by a user's operation, and the position of one or both of the left end (boundary line) and the right end (boundary line) of the highlight scene range frame 163 can be changed. As a result, in the highlight scene setting unit 53 of the image processing device 31, the range of the imaging time of the thumbnail included in the changed highlight scene range frame 163 is set as the time range of the highlight scene of the user setting, and the video data of the time range is uploaded to the cloud 14. Note that the highlight scene range frame 163 may be changed by sound. For example, the left end boundary of the highlight scene range frame 163 may be moved to the position of the thumbnail 10 minutes before on the basis of a sound such as “shift the start time 10 minutes before”. Furthermore, the highlight scene range frame 163 may be changed to a range including the thumbnail of the scene corresponding to the audio keyword on the basis of the audio keyword in association with scene understanding by video analysis. Furthermore, the highlight scene range frame 163 may be changed by the user's line of sight, operation of a foot switch, or the like.

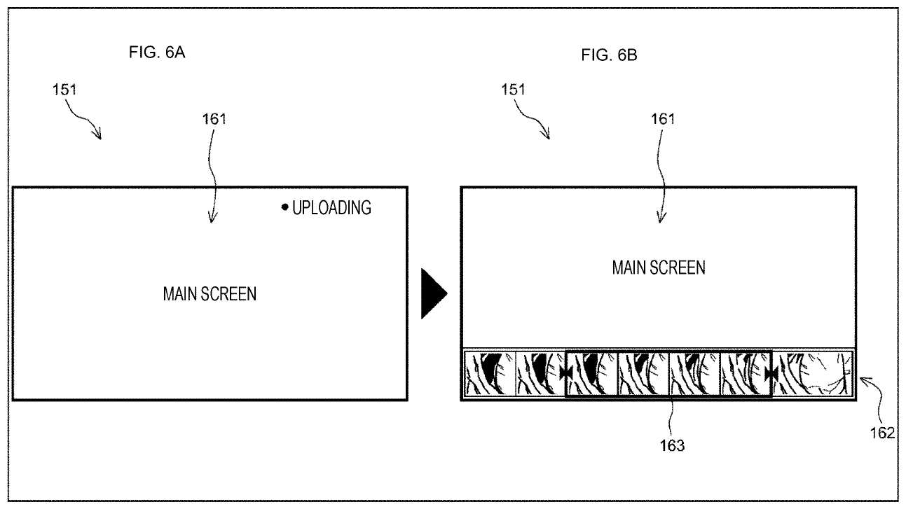

FIG. 6 is an application example of the first form of the video observation screen of FIG. 5. Note that, in the drawing, portions corresponding to those of the fifth video observation screen are denoted by the same reference signs, and the description thereof will be omitted. A video observation screen 151 in (A) of FIG. 6 is a screen displayed when video data captured by the camera 11 is uploaded to the cloud 14. Accordingly, when the video data is being uploaded, the characters “uploading” on the main screen 161 are displayed, and the highlight scene editing screen 162 is not displayed. A video observation screen 151 in (B) of FIG. 6 is a screen displayed when the video data is not uploaded to the cloud 14. The video observation screen 151 in (B) of FIG. 6 is the same as the video observation screen 151 in FIG. 5, and the user can perform an operation of changing the highlight scene range. Note that information notifying that the video displayed on the main screen is the video of the highlight scene may be displayed on the video observation screen 151 without being limited to the case where the upload is performed. The upload and the notification of the highlight scene are not limited to the case of character information.

### Second Form of Video Observation Screen

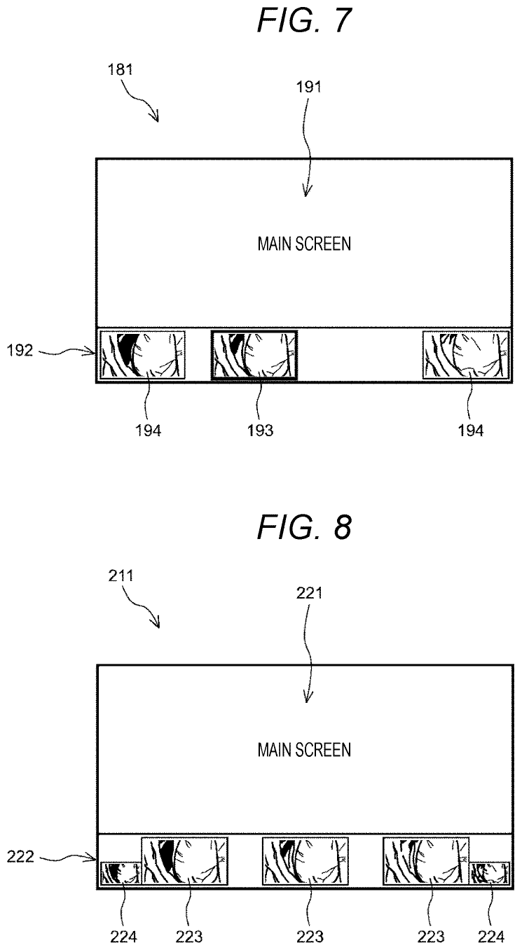

FIG. 7 is a diagram illustrating a second form of the video observation screen generated by the display control unit 56 of the image processing device 31 in FIG. 3 and displayed on the display unit 91 of the terminal device 32. In FIG. 7, a video observation screen 181 of the second form includes a main screen 191 and a highlight scene editing screen 192. A real-time video captured by the camera 11 is displayed on the main screen 191. On the highlight scene editing screen 192, thumbnails of images of frames at regular intervals in the video captured by the camera 11 or thumbnails representative for each scene are displayed in order from left to right in chronological order of the imaging time. On the highlight scene editing screen 192, the thumbnail 193 of the video included in the highlight scene range (the thumbnail 193 of the highlight scene range) is displayed in a form different from the thumbnail 194 of the non-highlight scene range not included in the highlight scene range (the thumbnail 194 of the non-highlight scene range). For example, in FIG. 7, the image frame of the thumbnail 193 of the highlight scene range is highlighted in the form of color, frame line width, and the like as compared with the thumbnail 194 of the non-highlight scene range. Furthermore, the thumbnail 193 of the highlight scene range may be displayed as a color image, and the thumbnail 194 of the non-highlight scene range may be displayed as a black-and-white image. The difference in display form between the thumbnail 193 of the highlight scene range and the thumbnail 194 of the non-highlight scene range may be in any display form as long as they can be identified. On the highlight scene editing screen 192, the range of thumbnails, which is the highlight scene range, is initially set on the basis of the time range of the highlight scene of the standard setting. On the other hand, for example, the user can switch the video of the thumbnail between the video of the highlight scene range and the video of the non-highlight scene range by performing an operation of designating a predetermined thumbnail (a touch operation or the like), whereby the user can change the highlight scene range and set the time range of the highlight scene of the user setting. The change of the highlight scene range may be performed using any method such as sound, line of sight, or foot switch similarly to the first form.

### Third Form of Video Observation Screen

FIG. 8 is a diagram illustrating a third form of the video observation screen generated by the display control unit 56 of the image processing device 31 in FIG. 3 and displayed on the display unit 91 of the terminal device 32. In FIG. 8, a video observation screen 211 of the third form includes a main screen 221 and a highlight scene editing screen 222. A real-time video captured by the camera 11 is displayed on the main screen 221. On the highlight scene editing screen 222, thumbnails of images of frames at regular intervals in the video captured by the camera 11 or thumbnails representative for each scene are displayed in order from left to right in chronological order of the imaging time. On the highlight scene editing screen 222, the thumbnail 223 of the video included in the highlight scene range (the thumbnail 223 of the highlight scene range) is displayed as a larger image than the thumbnail 224 of the non-highlight scene range not included in the highlight scene range (the thumbnail 224 of the non-highlight scene range). Furthermore, the thumbnail 223 of the highlight scene range may be displayed as a color image, and the thumbnail 224 of the non-highlight scene range may be displayed as a black-and-white image. On the highlight scene editing screen 222, the range of thumbnails, which is the highlight scene range, is initially set on the basis of the time range of the highlight scene of the standard setting. On the other hand, for example, the user can switch the video of the thumbnail between the video of the highlight scene range and the video of the non-highlight scene range by performing an operation of designating a predetermined thumbnail (a touch operation or the like). Thereby, the user can change the highlight scene range and set the time range of the highlight scene of the user setting. The change of the highlight scene range may be performed using any method such as sound, line of sight, or foot switch similarly to the first form.

### Fourth Form of Video Observation Screen

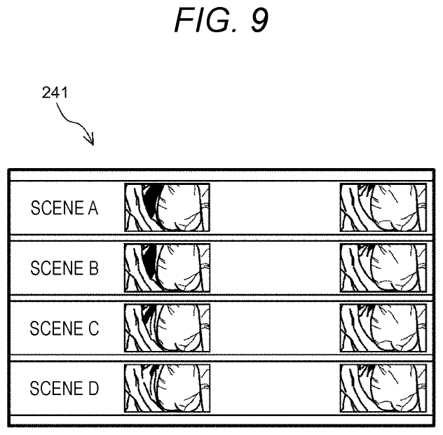

FIG. 9 is a diagram illustrating a fourth form of the video observation screen generated by the display control unit 56 of the image processing device 31 in FIG. 3 and displayed on the display unit 91 of the terminal device 32. In FIG. 9, a video observation screen 241 of the third form includes only a highlight scene editing screen. On the video observation screen 241, a list of scenes detected by the scene detection unit 52 of the image processing device 31 is displayed as selection buttons such as scene A, scene B, scene C, and scene D, which are identification information of scenes and representative thumbnails. On the video observation screen 241, initially, a scene selection button to be a highlight range is displayed on the basis of the time range of the highlight scene of the standard setting. On the other hand, the user can switch the video of the scene between the video of the highlight scene and the video of the non-highlight scene by performing a specification operation (a touch operation or the like) on the selection button of the desired scene, whereby the user can change the highlight scene range and set the time range of the highlight scene of the user setting. Note that the user's operation may be performed by voice. In the third form, a highlight scene can be selected only by specifying information for identifying a scene (scene A or the like) by voice. The selection of the highlight scene may be performed using any method such as a line of sight and a foot switch similarly to the first form.

### Configuration Example of Computer

A series of processing in the image processing device 31 and the like described above can be executed by hardware or software. In a case where a series of processing is executed by software, a program included in the software is installed on a computer. Here, examples of the computer include a computer incorporated in dedicated hardware, and for example, a general-purpose personal computer capable of executing various functions by installing various programs or the like.

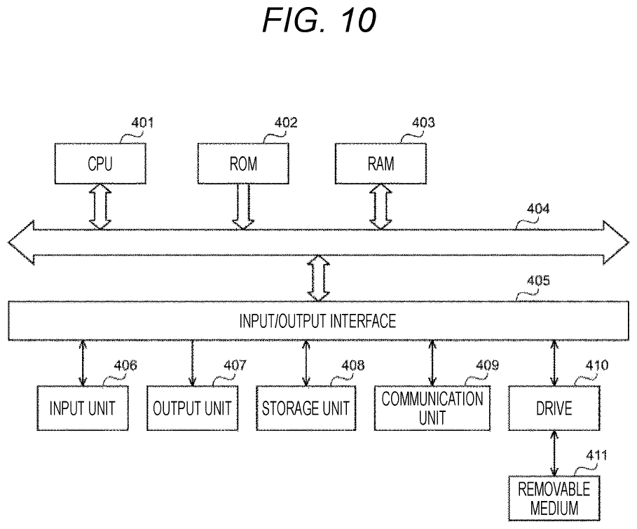

FIG. 10 is a block diagram illustrating a configuration example of hardware of a computer that executes the above-described series of processing by a program.

In the computer, a central processing unit (CPU) 401, a read only memory (ROM) 402, and a random access memory (RAM) 403 are mutually connected by a bus 404.

An input/output interface 405 is further connected to the bus 404. An input unit 406, an output unit 407, a storage unit 408, a communication unit 409, and a drive 410 are connected to the input/output interface 405.

The input unit 406 includes a keyboard, a mouse, a microphone, and the like. The output unit 407 includes a display, a speaker, and the like. The storage unit 408 includes a hard disk, a nonvolatile memory, and the like. The communication unit 409 includes a network interface and the like. The drive 410 drives a removable medium 411 such as a magnetic disk, an optical disk, a magneto-optical disk, a semiconductor memory, or the like.

In the computer configured as described above, for example, the CPU 401 loads a program stored in the storage unit 408 into the RAM 403 via the input/output interface 405 and the bus 404 and executes the program, so that the above-described series of processing is performed.

The program executed by the computer (CPU 401) can be provided by being recorded in the removable medium 411 as a package medium or the like, for example. Also, the program can be provided via a wired or wireless transmission medium such as a local area network, the Internet, or digital satellite broadcasting.

In the computer, the program can be installed in the storage unit 408 via the input/output interface 405 by attaching the removable medium 411 to the drive 410. Furthermore, the program can be received by the communication unit 409 via a wired or wireless transmission medium and installed in the storage unit 408. In addition, the program can be installed in the ROM 402 or the storage unit 408 in advance.

Note that the program executed by the computer may be a program in which processing is performed in time series in the order described in the present specification or may be a program in which processing is performed in parallel, or at a necessary timing such as when a call is made.

Here, in the present specification, the processing to be performed by the computer in accordance with the program is not necessarily performed in time series according to orders described in the flowcharts. That is, the processing to be performed by the computer in accordance with the program includes processing to be executed in parallel or independently of one another (parallel processing or object-based processing, for example).

Furthermore, the program may be processed by one computer (one processor) or processed in a distributed manner by a plurality of computers. Moreover, the program may be transferred to a distant computer to be executed.

Moreover, in the present specification, a system means a set of a plurality of components (devices, modules (parts), and the like), and it does not matter whether or not all the components are in the same housing. Therefore, a plurality of devices housed in separate housings and connected to each other via a network and one device in which a plurality of modules is housed in one housing are both systems.

Furthermore, for example, a configuration described as one device (or processing unit) may be divided and configured as a plurality of devices (or processing units). Conversely, configurations described above as a plurality of devices (or processing units) may be collectively configured as one device (or processing unit). Furthermore, a configuration other than the above-described configuration may be added to the configuration of each device (or each processing unit). Furthermore, as long as the configuration and operation of the entire system are substantially the same, a part of the configuration of a certain device (or processing unit) may be included in the configuration of another device (or another processing unit).

Furthermore, for example, the present technology can be configured as cloud computing in which one function is shared and jointly processed by the plurality of devices through the network.

Furthermore, for example, the program described above can be executed by any device. In this case, the device is only required to have a necessary function (functional block and the like) and obtain necessary information.

Furthermore, for example, each step described in the flowcharts described above can be executed by one device, or can be executed in a shared manner by the plurality of devices. Moreover, in a case where a plurality of processes is included in one step, the plurality of processes included in the one step can be executed by one device or shared and executed by the plurality of devices. In other words, the plurality of processes included in one step can also be executed as processes of a plurality of steps. Conversely, the processes described as the plurality of steps can also be collectively executed as one step.

Note that, in the program to be executed by the computer, the processes in steps describing the program may be executed in time series in the order described in the present specification, or may be executed in parallel, or independently at a necessary timing such as when a call is made. That is, unless there is a contradiction, the process in each step may also be executed in an order different from the orders described above. Moreover, the processes in the steps describing the program may be executed in parallel with processes of another program, or may be executed in combination with processes of the other program.

Note that, the plurality of present technologies that has been described in the present specification can each be implemented independently as a single unit unless there is a contradiction. It goes without saying that any plurality of present technologies can be implemented in combination. For example, some or all of the present technology described in any of the embodiments can be implemented in combination with some or all of the present technology described in other embodiments. Furthermore, some or all of the above-described arbitrary present technology can be implemented in combination with other technologies not described above.

### Combination Example of Configuration

Note that the present technology can also have the following configurations.
(1)

An information processing system including:
an acquisition unit that acquires a medical video captured by a medical image capturing device;
a setting unit that sets a highlight scene that is a candidate to be preferentially uploaded to a storage on the basis of the medical video;
a display control unit that generates a video of a first screen representing a range of an image included in the highlight scene on an arrangement screen in which images of frames at regular intervals in the medical video or images representative for each scene are arranged in chronological order; and
a change unit that changes the highlight scene on the basis of a user operation.
(2)

The information processing system according to (1),
in which the display control unit generates a video of a combined screen obtained by combining the first screen and a second screen for displaying the medical video.
(3)

The information processing system according to (1),
in which the display control unit generates a video of a combined screen obtained by combining the first screen and a second screen that displays the medical video in real time captured by the medical image capturing device.
(4)

The information processing system according to any one of (1) to (3),
in which the display control unit arranges the images of the frames or the image representative for each scene on the first screen by reduced images.
(5)

The information processing system according to any one of (1) to (4),
in which the change unit changes the highlight scene on the basis of the user operation to change a range of the image included in the highlight scene on the first screen generated by the display control unit.
(6)

The information processing system according to any one of (1) to (5),
in which the display control unit displays a boundary line between the highlight scene and a non-highlight scene on the arrangement screen in the first screen.
(7)

The information processing system according to any one of (1) to (6),
in which the setting unit sets the highlight scene as a candidate to be uploaded to the storage preferentially with respect to a resolution height.
(8)

The information processing system according to any one of (1) to (7),
in which the setting unit sets the highlight scene that is a candidate to be uploaded to the storage preferentially with respect to an order of upload.
(9)

The information processing system according to any one of (1) to (7),
further including
a processing unit that uploads a medical video included in the highlight scene set by the setting unit and a medical video included in the highlight scene changed by the change unit to the storage,
in which the processing unit uploads the medical video included in the highlight scene set by the setting unit to the storage before the change unit changes the highlight scene.
(10)

The information processing system according to (8),
in which the processing unit deletes the medical video included in the highlight scene set by the setting unit from the storage, and uploads the medical video included in the highlight scene changed by the change unit to the storage.
(11)

The information processing system according to (9),
in which the processing unit deletes, from the storage, a medical video not included in the highlight scene changed by the change unit among medical videos included in the highlight scene set by the setting unit, and uploads the medical video included in the highlight scene changed by the change unit to the storage.
(12)

The information processing system according to any one of (1) to (11),
further including
a processing unit that uploads a medical video included in the highlight scene set by the setting unit and a medical video included in the highlight scene changed by the change unit to the storage,
in which the processing unit uploads a medical video not included in the highlight scene to the storage as a video having a resolution lower than that of the medical video included in the highlight scene.
(13)

The information processing system according to any one of (1) to (12),
further including
a processing unit that uploads a medical video included in the highlight scene set by the setting unit and a medical video included in the highlight scene changed by the change unit to the storage,
in which the processing unit uploads information specifying a range of the medical video to be uploaded to the storage in the medical video captured by the medical image capturing device to the storage.
(14)

The information processing system according to any one of (1) to (13),
in which the change unit detects the user operation by a voice, a line of sight, or a foot switch.
(15)

An information processing method
including
an acquisition unit, a setting unit, a display control unit, and a change unit,
in which, in the information processing system,
the acquisition unit acquires a medical video captured by a medical image capturing device,
the setting unit sets a highlight scene that is a candidate to be preferentially uploaded to a storage on the basis of the medical video,
the display control unit generates a video of a first screen representing a range of an image included in the highlight scene on an arrangement screen in which images of frames at regular intervals in the medical video or images representative for each scene are arranged in chronological order, and
the change unit changes the highlight scene on the basis of a user operation.
(16)

A program for causing
a computer to function as:

an acquisition unit that acquires a medical video captured by a medical image capturing device;
a setting unit that sets a highlight scene that is a candidate to be preferentially uploaded to a storage on the basis of the medical video;
a display control unit that generates a video of a first screen representing a range of an image included in the highlight scene on an arrangement screen in which images of frames at regular intervals in the medical video or images representative for each scene are arranged in chronological order; and
a change unit that changes the highlight scene on the basis of a user operation.

### REFERENCE SIGNS LIST

1 Information processing system
11 Camera
12 In-hospital storage
13 IP network
14 Cloud
31 Image processing device
32, 33 Terminal device
51 Video acquisition unit
52 Scene detection unit
53 Highlight scene setting unit
54 Highlight scene extraction unit
55 Storage processing unit
56 Display control unit
57 Highlight scene change unit
71 Storage
91 Display unit
92 Input unit
111 Display unit
112 Input unit

1. An information processing system comprising:
an acquisition unit that acquires a medical video captured by a medical image capturing device;
a setting unit that sets a highlight scene that is a candidate to be preferentially uploaded to a storage on a basis of the medical video;
a display control unit that generates a video of a first screen representing a range of an image included in the highlight scene on an arrangement screen in which images of frames at regular intervals in the medical video or images representative for each scene are arranged in chronological order; and
a change unit that changes the highlight scene on a basis of a user operation.

2. The information processing system according to claim 1,
wherein the display control unit generates a video of a combined screen obtained by combining the first screen and a second screen for displaying the medical video.

3. The information processing system according to claim 1,
wherein the display control unit generates a video of a combined screen obtained by combining the first screen and a second screen that displays the medical video in real time captured by the medical image capturing device.

4. The information processing system according to claim 1,
wherein the display control unit arranges the images of the frames or the image representative for each scene on the first screen by reduced images.

5. The information processing system according to claim 1,
wherein the change unit changes the highlight scene on a basis of the user operation to change a range of the image included in the highlight scene on the first screen generated by the display control unit.

6. The information processing system according to claim 1,
wherein the display control unit displays a boundary line between the highlight scene and a non-highlight scene on the arrangement screen in the first screen.

7. The information processing system according to claim 1,
wherein the setting unit sets the highlight scene as a candidate to be uploaded to the storage preferentially with respect to a resolution height.

8. The information processing system according to claim 1,
wherein the setting unit sets the highlight scene that is a candidate to be uploaded to the storage preferentially with respect to an order of upload.

9. The information processing system according to claim 1,
further comprising
a processing unit that uploads a medical video included in the highlight scene set by the setting unit and a medical video included in the highlight scene changed by the change unit to the storage,
wherein the processing unit uploads the medical video included in the highlight scene set by the setting unit to the storage before the change unit changes the highlight scene.

10. The information processing system according to claim 9,
wherein the processing unit deletes the medical video included in the highlight scene set by the setting unit from the storage, and uploads the medical video included in the highlight scene changed by the change unit to the storage.

11. The information processing system according to claim 9,
wherein the processing unit deletes, from the storage, a medical video not included in the highlight scene changed by the change unit among medical videos included in the highlight scene set by the setting unit, and uploads the medical video included in the highlight scene changed by the change unit to the storage.

12. The information processing system according to claim 1,
further comprising
a processing unit that uploads a medical video included in the highlight scene set by the setting unit and a medical video included in the highlight scene changed by the change unit to the storage,
wherein the processing unit uploads a medical video not included in the highlight scene to the storage as a video having a resolution lower than that of the medical video included in the highlight scene.

13. The information processing system according to claim 1,
further comprising
a processing unit that uploads a medical video included in the highlight scene set by the setting unit and a medical video included in the highlight scene changed by the change unit to the storage,
wherein the processing unit uploads information specifying a range of the medical video to be uploaded to the storage in the medical video captured by the medical image capturing device to the storage.

14. The information processing system according to claim 1,
wherein the change unit detects the user operation by a voice, a line of sight, or a foot switch.

15. An information processing method
comprising
an acquisition unit, a setting unit, a display control unit, and a change unit,
wherein, in the information processing system,
the acquisition unit acquires a medical video captured by a medical image capturing device,
the setting unit sets a highlight scene that is a candidate to be preferentially uploaded to a storage on a basis of the medical video,
the display control unit generates a video of a first screen representing a range of an image included in the highlight scene on an arrangement screen in which images of frames at regular intervals in the medical video or images representative for each scene are arranged in chronological order, and
the change unit changes the highlight scene on a basis of a user operation.

16. A program for causing
a computer to function as:
an acquisition unit that acquires a medical video captured by a medical image capturing device;
a setting unit that sets a highlight scene that is a candidate to be preferentially uploaded to a storage on a basis of the medical video;
a display control unit that generates a video of a first screen representing a range of an image included in the highlight scene on an arrangement screen in which images of frames at regular intervals in the medical video or images representative for each scene are arranged in chronological order; and
a change unit that changes the highlight scene on a basis of a user operation.
